(19) United States
(12) Patent Application Publication
(10) Pub. No.: US 2023/0241215 A1
(43) Pub. Date: Aug. 3, 2023

(54) USE OF LOW IRON OXIDE IRON-DOPED TITANIUM DIOXIDE NANOPARTICLES IN THE TREATMENT OF TUMORS AND OTHER DISEASES

(71) Applicant: Rodney HERRING, Victoria, BC (CA)

(72) Inventor: Rodney HERRING, Victoria, BC (CA)

(21) Appl. No.: 18/002,852

(22) PCT Filed: Jun. 24, 2021
(86) PCT No.: PCT/CA2021/050875

§ 371 Date: Dec. 21, 2022

(30) Foreign Application Priority Data
Jun. 24, 2020 (CA) 3084780

(51) Int. Cl. A61K 41/00; A61K 9/51; A61K 9/50; A61K 9/00; A61N 1/04; A61B 18/14

(52) U.S. Cl. CPC A61K 41/0047; A61K 9/5115; A61K 9/501; A61K 9/0009; A61N 1/0484; A61B 18/1402; A61B 2018/00577

(57) ABSTRACT

A kit for electrocatalytically treating a target tissue is provided, the kit comprising: substantially iron oxide free iron-doped titanium dioxide nanoparticles; a voltage generator; and at least one electrode pair consisting of an anode and a cathode, the electrode pair for electrical communication with the voltage generator. A use of the kit is also provided.

### FIELD

The present technology is directed to a kit comprising an electrical therapy apparatus and low iron oxide, iron-doped titanium dioxide nanoparticles for the treatment of target tissue such as tumors. More specifically, it directed to the use of the kit in the treatment of a patient in need thereof.

### BACKGROUND

In recent years, many studies have also focused on the biomedical applications of titanium dioxide nanoparticles using UV light to activate the nanoparticles. For example, “The Photocatalytic Inactivation Effect of Fe-Doped TiO2 Nanocomposites on Leukemic HL60 Cells-Based Photodynamic Therapy” (International Journal of Photoenergy Volume 2012, Article ID 367072) discloses: The experimental results showed that the growth of leukemic HL60 cells was significantly inhibited by adding TiO2 nanoparticles, and the inactivation efficiency could be effectively enhanced by the surface modification of TiO2 nanoparticles with Fe doping. Furthermore, the optimized conditions were achieved at Swt % Fe/TiO2 at a final concentration of 200 μg/mL, in which up to 82.5% PDT efficiency for the HL60 cells can be obtained under the irradiation of 403 nm light (the power density is 5 mW/cm2) within 60 minutes. If this were to be used to treat a patient, it would necessarily be invasive in order to get the light to the nanoparticles.

Other approaches include electrochemical treatment (ECT). In the ECT technique disclosed by Li et al., in Bioelectromagnetics 18:2-7 (1997), in an article “Effects of Direct Current on Dog Liver: Possible Mechanisms For Tumor Electrochemical Treatment” two platinum anode and cathode electrodes were inserted in a dog's liver with a 3 cm separation therebetween. A direct current voltage of 8.5 volts was applied across the electrodes, giving rise to an average current through the liver of 30 mA. The series of electrochemical reactions which took place during ECT resulted in the rapid and complete destruction of both normal and tumor cells in the liver. This is an invasive method, with a very high amperage.

Another example of ECT appears in the article “Electrochemical Treatment of Lung Cancer” by Xin et al. in Bioelectromagnetics 18:8-13 (1997). In this ECT procedure platinum electrodes were inserted transcutaneously into the tumor, the voltage applied thereto being in the 6-8 volt range, the current being in the 40 to 100 mA range, and the electric charge, 100 coulombs per cm of tumor diameter.

Electrochemical reactions as a function of pH and electrode potential can be predicted by means of a Pourbaix diagram, as disclosed in the Atlas of Electrochemical Equilibria in Aqueous Solutions—Pergamon Press, 1966—by Pourbaix. Reaction products of electrolysis of water include hydrogen, oxygen, and hydrogen peroxide. In the text Methods in Cell Biology, Vol. 46—Cell Death—published by Academic Press, 1995, it is noted (page 163), that hydrogen peroxide has been reported to be an inducer of cell death in various cell systems. This type of cell death is attributed to the direct cytotoxicity of hydrogen peroxide and other oxidant species generated from hydrogen peroxide.

United States Patent Application No. 20120165186 discloses a process for producing a concentrated aqueous nano titania sol in the mild pH range (4.0 to 10.0) comprising contacting an acidic nano titania sol with a dispersant and with an alkalizing agent, and subjecting the nano titania sol to membrane filtration until the nano titania sol contains more than 300 g TiO.sub.2 nanoparticles/dm.sup.3. The nano titania sol may further be subjected to a coating treatment within any of the steps of the above described process.

The concentrated aqueous nano titania sol of this disclosure is suitable for use in a variety of applications, including providing UV protection and photochemically degrading or inactivating contaminants. The catalytic composition may be used as an anti-cancer agent when delivered to tumor cells. It may be desirable to couple the catalytic composition to a targeting agent that is selectively absorbed by tumor cells. Light may be delivered to the cells containing the catalytic composition Iaparoscopically, resulting in cell death or a reduction in cell growth or propagation. Thus, the method is invasive.

Electrochemical methods have also been disclosed for treating tumors. For example, U.S. Pat. Nos. 7,526,334 and 6,708,066 disclose a technique and apparatus therefor adapted to treat in situ specified tissue, especially a malignant tumor, use being made of electrodes implanted in the tissue at spaced positions. Applied across the electrodes is a voltage causing a current to flow through the tissue to be treated. This current in one embodiment of the invention produces an electrochemical reaction yielding multiple reaction products, some of which are cytotoxic agents destructive of cancer cells, the voltage being regulated to optimize the yield of those agents having the greatest efficacy. In another embodiment, fed to the tissue is one or more reagents which when current flows through the tissue react with the material of an electrode to yield a cytotoxic agent in situ. Alternatively, the surface of the electrode can serve as a catalyst for the formation of the cytotoxic agents. Metal complexes of titanium are disclosed as a potential electrode for use with a substance that reacts with the metal to produce a cytotoxic agent. This, therefore, requires an additional substance in order to produce the cytotoxic agent. The method is invasive.

What is needed is a non-invasive method of treating tumors. More specifically, there is a need for non-invasive treatment of cancerous tumors. It would be preferable if the treatment minimally harmed surrounding tissue. It would be preferable if the substance used in the treatment was safe to ingest or inject and had no side effects. It would be preferable if the substance used in the treatment selectively accumulated in metabolically active tissues, such as tumors and other diseased tissue, without the need for it to be coupled to a targeting agent. It would be preferable if a kit was provided for the treatment.

### SUMMARY

The present technology is a non-invasive method of treating a target tissue such as tumors. More specifically, the method is for non-invasive treatment of cancerous tumors. The method minimally harms surrounding tissue. The substance used in the treatment is safe to ingest or inject and has minimal side effects. The substance, substantially iron oxide free iron-doped titanium dioxide, selectively accumulates in metabolically active tissues, such as tumors and other diseased tissue. The substantially iron oxide free iron-doped titanium dioxide does not need to be coupled to a targeting agent in order to selectively accumulate in the target tissue. A kit is provided for the treatment.

In one embodiment, a kit for electrocatalytically treating a target tissue is provided, the kit comprising: nanoparticles having a bandgap energy of about 2.5 electron volts to about 40 electron volts; a voltage generator; and at least one electrode pair consisting of an anode and a cathode, the electrode pair for electrical communication with the voltage generator.

In the kit, the voltage generator may include a voltage adjustment dial.

In the kit, the voltage generator may be configured to provide a voltage of about 3 volts to about 40 volts.

In the kit, the voltage generator may be configured to produce an amperage of about 1 to about 4 milliamps.

In the kit, the anode and cathode may be ear clips.

In the kit, the anode and the cathode may be electrode pads.

The kit may further comprise an electrode gel.

In the kit, there may be a plurality of electrode pairs.

The kit may further comprise a potable liquid, an edible gel or an edible cream that includes the nanoparticles.

The kit may further comprise a diluent that includes the nanoparticles.

In the kit, the nanoparticles may be nanoparticles are substantially iron oxide free iron-doped titanium dioxide nanoparticles.

In another embodiment, a use of the kit on a patient in need thereof is provided.

### FIGURES

FIG. 1 is a schematic of the kit of the present technology.

FIG. 2 is a schematic of the method of the present technology.

### DESCRIPTION

Except as otherwise expressly provided, the following rules of interpretation apply to this specification: (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Thin film—in the context of the present technology, a thin film is up to 5 microns in thickness. A film may be a partial coating, a deposit upon a surface, a complete coating or a plurality of layers. To be clear, gaps may occur where the surface below is exposed. It may be formed by, for example, but not limited to growing nanocrystals on the substrate, physical vapour deposition on the substrate or photolithography on the substrate.

Iron-doped titanium dioxide with a low iron oxide surface—in the context of the present technology, iron-doped titanium dioxide with a low iron oxide surface has about 0.1 atomic % iron to about 2.0 atomic % iron, preferably 0.25 atomic % iron to about 0.75 atomic % iron, and more preferably 0.5 atomic % iron and very small amounts of iron oxide on its surface (less than 5% of the surface being iron oxide) when viewed with X-ray photoelectron spectroscopy.

Substantially iron oxide free surface—in the context of the present technology, a substantially iron oxide free surface has an iron oxide content corresponding to less than about 0.001% atomic iron (less than 0.5% of the surface being iron oxide) when viewed with X-ray photoelectron spectroscopy.

### DETAILED DESCRIPTION

The catalysts were prepared by the sol-gel method using titanium isopropoxide (TTIP) as the precursor and ferric nitrate (Fe(NO₃)3.9H₂O) as the iron source. Firstly, the desired amount of ferric nitrate (0.25, 0.5, 1, 5 and 10 molar %) was dissolved in water and then the solution was added to 30 mL of anhydrous ethyl alcohol and stirred for 10 minutes. The acidity of the solution was adjusted to about pH 3 (about pH 2.5 to about pH 3.5) using HNO3(other acids could also be used), which produces better Fe doped TiO₂, i.e., incorporation of Fe into the TiO₂ nanocrystals. Secondly, TTIP was added dropwise to the solution. Then deionized water with the ratio of Ti:H₂O (1:4) was added to the mixture.

The solution was stirred for two hours and then dried at 80° C. for two hours.

The powders were then washed three times with deionized water. Next, the powder was calcined at 400° C. for three hours. To compare the influence of acid washing on the photocatalytic performance of the calcined powder, a portion of it was stirred in an HCl solution (acid washed) and then washed with deionized water three times. The acid washing was in a solution of about pH 2.5 to about pH 3.5, or about pH 4, with, preferably, a monoprotic acid, such as, for example, but not limited to acetic acid (CH₃CO₂H or HOAc), hydrochloric acid (HCl), hydroiodic acid (HI), hydrobromic acid (HBr), perchloric acid (HClO₄), nitric acid (HNO₃) or sulfuric acid (H₂SO₄), with HCl being the preferred.

A second method of preparing the low iron oxide, iron-doped titanium dioxide functionalized fiberglass or sintered glass is as follows:

The low iron oxide, iron-doped titanium dioxide nanoparticles were prepared by the sol-gel method using titanium isopropoxide (TTIP) as the precursor and ferric nitrate (Fe(NO₃)3.9H₂O) as the iron source. Firstly, the desired amount of ferric nitrate (0.25, 0.5, 1, 5 and 10 molar %) was dissolved in water and then the solution was added to 30 mL of anhydrous ethyl alcohol and stirred for 10 minutes. The acidity of the solution was adjusted to about pH 3 (about pH 2.5 to about pH 3.5) using HNO₃ (other acids could also be used), which produces better Fe doped TiO₂, i.e., incorporation of Fe into the TiO₂ nanoparticles. Secondly, TTIP was added dropwise to the solution. Then deionized water with the ratio of Ti:H₂O (1:4) was added to the mixture. The solution was stirred for two hours and then dried at 80° C. for two hours.

The powders were then washed three times with deionized water. Next, the powder was calcined at 400° C. for three hours. The calcined powder was stirred in an HCl solution (acid washed) and then washed with deionized water three times. The acid washing was in a solution of about pH 2.5 to about pH 3.5, or about pH 4, with, preferably, a monoprotic acid, such as, for example, but not limited to acetic acid (CH₃CO₂H or HOAc), hydrochloric acid (HCl), hydroiodic acid (HI), hydrobromic acid (HBr), perchloric acid (HClO₄), nitric acid (HNO₃) or sulfuric acid (H₂SO₄), with HCl being the preferred. The acid washing produced low iron oxide, iron-doped titanium dioxide.

Regardless of the method of producing the low iron oxide, iron-doped titanium dioxide nanoparticles, the acid washing was shown to remove a significant amount of iron oxide from the surface of the nanoparticles. The acid-washed iron-doped titanium dioxide nanoparticles function as electrocatalysts.

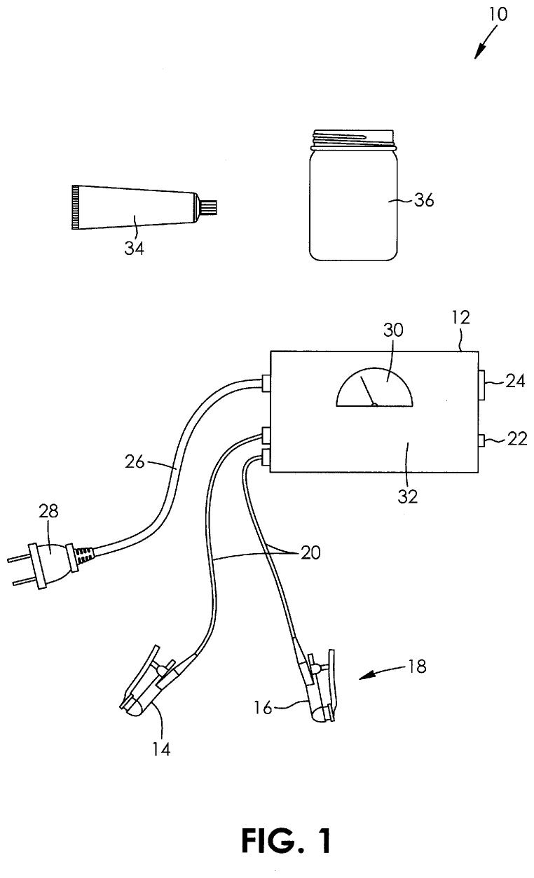

A kit, generally referred to as 10 is shown in FIG. 1. The kit 10 includes a voltage generator 12 which provides electricity to two electrodes, an anode 14 and a cathode 16, which in one embodiment are ear clips, generally referred to as 18. The voltage generator 12 is connected to the ear clips 18 with lead wires 20. The lead wires 20 are releasably attached to the voltage generator 12 in order to allow for other electrodes to be employed as described below. The device 10 includes an ON/OFF switch 22, and a dial 24 that controls the voltage output. A power cord 26 includes a plug 28 for plugging the device 10 into an outlet. A voltmeter 30 on the front 32 of the device shows the voltage being applied. A tube 30 of electrode gel, such as Spectra® 360, is provided in the kit 10. A jar 32 of low iron oxide, iron-doped titanium dioxide nanoparticles is also provided in the kit 10. In one embodiment, the low iron oxide, iron-doped titanium dioxide nanoparticles have surfaces that are substantially iron oxide-free. In one embodiment, the low iron oxide, iron-doped titanium dioxide nanoparticles are provided in a potable liquid or edible gel or edible cream and the like.

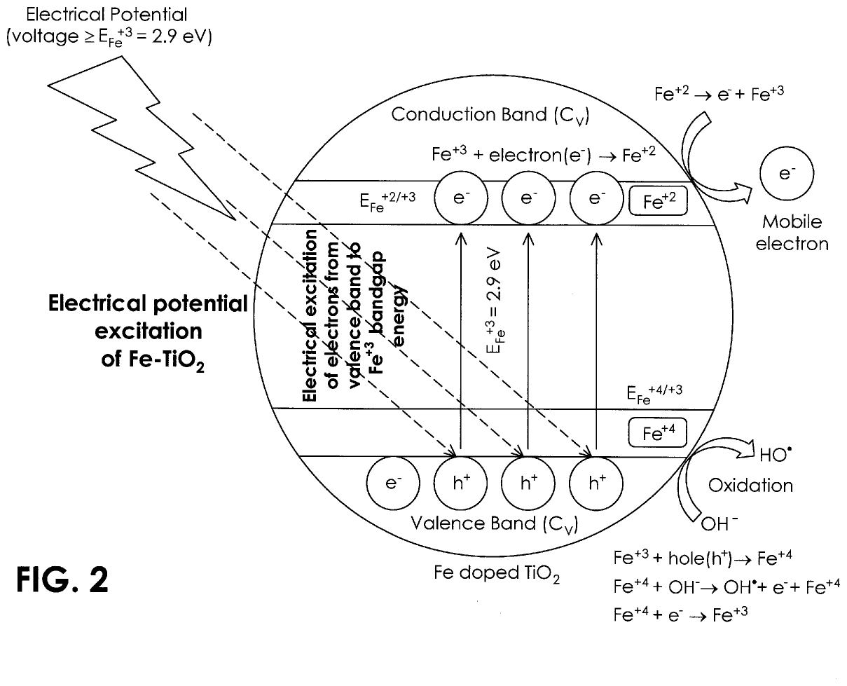

In the use of the kit, a person ingests a source of low iron oxide, iron-doped titanium dioxide nanoparticles. The source may be for example, but not limited to, a food thickened with the low iron oxide, iron-doped titanium dioxide nanoparticles or a drink that includes the low iron oxide, iron-doped titanium dioxide nanoparticles. The nanoparticles pass through the intestine into the blood where they are transported to high metabolic areas of the body such as diseased areas or tumors. The nanoparticles easily pass through the blood brain barrier. The ear clips are clipped on the person's ears, one per ear. The device is turned on, the voltage is set and current flows to the anode, through the person's brain, to the cathode, intercepting the low iron oxide, iron-doped titanium dioxide nanoparticles. The voltage ranges between about 3 volts to about 40 volts, with 20 volts being preferred and the amperage is about 1 to about 4 milliamps. FIG. 2 shows how the method is effected.

In another embodiment, in the use of the kit, the person is injected with a source of low iron oxide, iron-doped titanium nanoparticles. The source may be saline or sterile water or other acceptable diluent with the low iron oxide, iron-doped titanium nanoparticles. In another embodiment, the anode 14 and the cathode 16 are electrode pads generally referred to as 118. The electrode pads 118 either include an electrode gel surface 120 or a tube 130 of electrode gel is provided. The electrode pads 118 are placed on the person's head such that the current passes through the tumor, hence one is on one side and the other is on the other, or one is on the front and the other is on the back.

In another embodiment, there are at least two anodes 114, 214 and at least two cathodes 116, 216. The anodes 114, 214 and the cathodes 116, 216 are electrode pads, generally referred to as 118. The electrode pads 118 either include an electrode gel surface 120 or a tube 130 of electrode gel is provided. The anode 114 and the cathode 116 of the first electrode pair are aligned such that the current passes through the tumor in one direction while the anode 214 and the cathode 216 of the second electrode pair are aligned such that the current passes through the tumor in a second direction. The anodes 114, 214 and cathodes 116, 216 can be attached to any part of the person's body for treatment of a tumor. The tumor may be ablated, eliminated or reduced in size.

The combination of strategic placement of the electrode pairs on the patient and the selective accumulation of the nanoparticles in tissues with high metabolic activity allow for directed, focused, non-invasive treatment of the target tissues. The in situ production of hydroxyl radicals leads to the ablation, elimination or reduction in size of the target tissue.

In another embodiment, colloidal carriers such as micelles, liposomes, and emulsions are used to increase the concentrations of the low iron oxide, iron-doped titanium dioxide nanoparticles in target tissue. The micelles and liposomes also control the release rate in the target tissue.

In yet another embodiment, targeting molecules are used to increase the concentrations of the low iron oxide, iron-doped titanium dioxide nanoparticles in target tissue.

In yet another embodiment, titanium dioxide nanoparticles (band gap energy of at least 3.2 electron volts) are used. The titanium dioxide nanoparticles are delivered to the patient either by ingestion or injection. The ear clips are clipped on the person's ears, one per ear. The device is turned on, the voltage is set and current flows to the anode, through the person's brain, to the cathode, intercepting the titanium dioxide nanoparticles. The voltage ranges between about 3 volts to about 40 volts, with 20 volts being preferred and the amperage is about 1 to about 4 milliamps.

In yet another embodiment, doped nanoparticles with a bandgap energy of about 2.5 electron volts to about 40 electron volts are used. These include, but are not limited to nitrogen-doped titanium dioxide nanoparticles (band gap energy of at least 2.5 electron volts), copper-doped titanium dioxide nanoparticles, chromium-doped titanium dioxide nanoparticles, manganese-doped titanium dioxide nanoparticles, cobalt-doped titanium dioxide nanoparticles or nickel-doped titanium dioxide nanoparticles are used. The nanoparticles are delivered to the patient either by ingestion or injection. The ear clips are clipped on the person's ears, one per ear or electrode pads are used. The device is turned on, the voltage is set and current flows to the anode, through the person's brain, to the cathode, intercepting the nanoparticles. The voltage ranges between about 3 volts to about 40 volts, with 20 volts being preferred and the amperage is about 1 to about 4 milliamps.

In yet another embodiment, other photocatalytic nanoparticles with a bandgap energy of about 3 electron volts to about 40 electron volts are used. These include, but are not limited to, zinc oxide (band gap energy of at least 3.29 electron volts), gold, silver, platinum, copper, silver-doped copper oxide, gold-doped copper oxide, silver-doped silicon dioxide and gold-doped silicon dioxide are used. The nanoparticles are delivered to the patient either by ingestion or injection. The ear clips are clipped on the person's ears, one per ear or electrode pads are used. The device is turned on, the voltage is set and current flows to the anode, through the person's brain, to the cathode, intercepting the nanoparticles. The voltage ranges between about 3 volts to about 40 volts, with 20 volts being preferred and the amperage is about 1 to about 4 milliamps.

Without being bound to theory, while the nanoparticles reside within the cell, if an electrical potential or voltage is applied that is greater than the energy required to stimulate (substantially iron oxide free) iron-doped titanium dioxide electrons from the valence band to the conduction band (>3 V) then positive charges or holes are created in the valence band of the iron doped nanoparticles. The conduction electrons and holes are captured by Fe+3 ions, which separates the ions so the electrons and holes don't recombine. The conduction electrons leave the nanoparticles to become part of the electric current that's passing from one electrode to another. The holes react with OH-ions in the cytoplasm to create hydroxyl radicals, OH:, that attack and kill the tumor cell.

The electrons removed from the valence band into the conduction band are replaced by an electron injected into the nanoparticles from the electric current passing between the electrodes. The electrons in the valence band are stimulated into the conduction band by the electric potential or voltage greater than the bandgap energy of Fe+3 ions creating positive holes in the valence band. The cycle repeats itself.

In all embodiments, if blood hypoxia conditions exist, the nanoparticles still produce hydroxyl radicals, which relies on the presence of hydroxyl ions present in the blood. There are 3×10exp(16) OH— ions in one mole of blood. The electric current passes through the blood because there's iron in it providing less resistance than passing through other body fluids. The nanoparticles are taken up by the cancer cells that are living in the blood vessels. The current preferentially passes through the nanoparticles taken up by the blood cells because there's less resistance to the current passing through the nanoparticles than the cytoplasm. The nanoparticles are a semiconductor (low resistance) whereas the cytoplasm is a dielectric fluid (high resistance). So, the nanoparticles are activated inside the cancer cells by the electric current producing hydroxyl radicals that kill the cancer.

While the technology has been described in detail, such a description is to be considered as exemplary and not restrictive in character and is to be understood that it is the presently preferred embodiments of the present technology and is thus representative of the subject matter which is broadly contemplated by the present technology, and that all changes and modifications that come within the spirit of the disclosure are desired to be protected.

1. A kit for electrocatalytically treating a target tissue, the kit comprising: nanoparticles selected from the group consisting of nitrogen-doped titanium dioxide nanoparticles, copper-doped titanium dioxide nanoparticles, chromium-doped titanium dioxide nanoparticles, manganese-doped titanium dioxide nanoparticles, cobalt-doped titanium dioxide nanoparticles, nickel-doped titanium dioxide nanoparticles and substantially iron oxide free titanium dioxide nanoparticles; a voltage generator; and at least one electrode pair consisting of an anode and a cathode, the electrode pair for electrical communication with the voltage generator.

2. The kit of claim 1, wherein the nanoparticles are selected from the group consisting of copper-doped titanium dioxide nanoparticles, chromium-doped titanium dioxide nanoparticles, manganese-doped titanium dioxide nanoparticles, cobalt-doped titanium dioxide nanoparticles, nickel-doped titanium dioxide nanoparticles and substantially iron oxide free titanium dioxide nanoparticles.

3. The kit of claim 2, wherein the voltage generator is configured to provide a voltage of about 3 volts to about 40 volts.

4. The kit of claim 2, wherein the voltage generator is configured to produce an amperage of about 1 to about 4 milliamps.

5. The kit of claim 4, wherein the anode and cathode are ear clips.

6. The kit of claim 4, wherein the anode and the cathode are electrode pads.

7. The kit of claim 6, further comprising an electrode gel.

8. The kit of claim 7, wherein there are a plurality of electrode pairs.

9. The kit of claim 8, further comprising a potable liquid, an edible gel or an edible cream that includes the nanoparticles.

10. The kit of claim 8, further comprising a diluent that includes the nanoparticles.

11. (canceled)

12. A use of the kit of claim 1 on a patient in need thereof.

13. A use of the kit of claim 2 on a patient in need thereof.

14. (canceled)

15. (canceled)
